(19) United States
(12) Patent Application Publication
(10) Pub. No.: US 2026/0083922 A1
(43) Pub. Date: Mar. 26, 2026

(54) Atomizing Body Assembly and Soft Mist Device with Improved Sealing Performance

(71) Applicant: Guangzhou Anobri Pharmaceutical Co. Ltd., Guangdong (CN)

(72) Inventors: Cai Gu Huang, Guangdong (CN); Shan Ming Chen, Guangdong (CN)

(21) Appl. No.: 19/106,729

(22) PCT Filed: Oct. 31, 2022
(86) PCT No.: PCT/CN2022/128511

§ 371 Date: Feb. 26, 2025

(30) Foreign Application Priority Data
Sep. 1, 2022 (CN) 202211064716.9

(51) Int. Cl. A61M 11/00; A61M 11/02

(52) U.S. Cl. CPC A61M 11/003; A61M 11/02

(57) ABSTRACT

The present application discloses an atomizing body assembly and a soft mist inhalation device with improved sealing performance. The atomizing body assembly includes a T-shaped steel member, a chip-mounting part, a filter cartridge, and a medium-sized O-ring. The chip-mounting part, the medium-sized O ring and the filter cartridge collectively form a sealing structure.

### CROSS-REFERENCE TO RELATED APPLICATIONS

This application is a national stage filing under 35 U.S.C. § 371 of international application number PCT/CN2022/128511, filed Oct. 31, 2022, which claims priority to Chinese patent application No. 202211064716.9, filed Sep. 1, 2022. The contents of these applications are incorporated herein by reference in their entireties.

### BACKGROUND OF THE INVENTION

A soft mist inhaler for administering a drug substance through inhalation can generate microdroplets by pushing, under a high pressure, a medicinal liquid through a microfluid channel structure included in an atomizing body assembly. Many of the micro droplets are sprayed out from the soft mist inhaler in the form of aerosols such that the drug substance can be inhaled. Aerosols that can be directly drawn into the lung have a size of 1-5 microns, and the fluid pressure required to convert the medicinal liquid into aerosols of 1-5 microns is between 5 Mpa to 40 MPa. Because a higher pressure demands a tighter sealing structure, studies have found that the existing sealing structure cannot meet this sudden change of the liquid pressure. The main problems are discussed below.

In an aspect, soft-mist inhalers are usually used for continuous treatment with 1-3 uses per day and 1-2 sprays of each use. However, some patients may not follow their doctor's advice and may use a soft-mist inhaler only when they have already developed symptoms of chronic obstructive pulmonary diseases, such as cough or asthma. When a soft mist inhaler is not used for 3 days or longer, the inhaler needs to be primed once or twice until it produces soft mist again for reuse, which causes the waste of the medicinal liquid and an inaccurate indication of the number of past uses.

In another aspect, only aerosols of 1-5 microns can be directly drawn into the lungs. But the microdroplets in the range of 1-5 microns generated by existing soft mist inhalers do not take a large portion of the generated aerosol. As the medicinal liquid discharged by a soft mist inhaler has large droplet sizes, they are not directly drawn into the lung, thus causing a low utilization rate of the medicinal fluid.

Through research and analysis, it has been found that the above problems are caused by the poor sealing performance of an atomizing body assembly of the soft mist inhaler. Thus, the improvement of the sealing performance of the atomizing body assembly becomes an urgent problem for the soft mist inhaler to be solved.

### SUMMARY OF THE INVENTION

The disclosure relates to an atomizing body assembly for improving sealing performance. The atomizing body assembly includes a T-shaped steel member, a chip-mounting part, a filter cartridge, and a medium-sized O-ring. The chip is mounted in the chip-mounting part. The top of the chip-mounting part is mounted in a receiving cavity of the T-shaped steel member such that the chip is firmly fixed and sealed in the chip-mounting part. The bottom of the chip-mounting part is in contact with the top of the filter cartridge. The medium-sized O-ring is disposed between the chip-mounting part and the filter cartridge, and the chip-mounting part, the medium-sized O-ring and the filter cartridge form a sealing structure.

Preferably, the T-shaped steel member includes a cylindrical housing structure. The interior of the T-shaped steel member includes a large conical surface, a small conical surface, a cylindrical surface, and a top surface, which are sequentially connected. The large conical surface, the small conical surface, the cylindrical surface, and the top surfaces form the receiving cavity for accommodating the top of the chip-mounting part.

Preferably, a chip-mounting part has a cylindrical structure with an annular structure protruding radially outward at the bottom.

Preferably, a top portion of the chip-mounting part is provided with a top surface. Starting from the top surface, a cylindrical surface, a small conical surface, a large conical surface, and a step surface are connected sequentially. The step surface corresponds to an upper annular surface of the annular structure protruding radially outward at the bottom of the chip-mounting part.

Preferably, the bottom portion of the T-shaped steel member is provided with an end surface and the end surface is pressed against the step surface of the chip-mounting part.

Preferably, a bottom annular surface of the annular structure protruding radially outward at the bottom of the chip-mounting part and a bottom end surface of the chip-mounting part are disposed on the same plane and form a bottom surface. The bottom surface is pressed against the top of the filter cartridge.

Preferably, a filter cartridge includes a cylindrical structure provided with an annular groove and a through-hole along the axis.

Preferably, a filter cartridge includes a top annular surface, a bottom annular surface, a top annular groove, a bottom annular groove, and the through-hole. The top annular groove is disposed on the top annular surface, and the bottom annular groove is disposed on the bottom annular surface.

Preferably, the top annular groove holds the medium-sized O-ring. The end face of the T-shaped steel member is pressed against the step surface of the chip-mounting part. The bottom face of chip-mounting part is pressed against the medium-sized O-ring and the top annular surface of the filter cartridge. The chip-mounting part and the medium-sized O-ring are deformed, thereby improving the sealing performance.

Preferably, a chip-mounting part is provided with a through-hole along the axis of the chip-mounting part. The through-hole includes a groove for accommodating the chip and a hole. The groove is in a fluid communication with the hole.

Preferably, the hole is a slot-shaped hole.

Preferably, the opening of the groove for the chip is provided with an inclined surface that facilitates a smooth insertion of the chip into the groove for accommodating the chip.

Preferably, the atomizing body assembly further comprises a large-sized O-ring, an atomizer body, and a large metal cap threadedly fixed to the atomizer body. The large metal cap is screwed on the atomizer body to form a receiving cavity for mounting the T-shaped steel member, the chip-mounting part, the chip, the filter cartridge, the medium-sized O-ring, and the large-sized O-ring. The internal side of the large metal cap is pressed against the top of the T-shaped steel member. The atomizer body is pressed against the bottom annular surface of the filter cartridge, and the large-sized O-ring is disposed in the bottom annular groove of the filter cartridge.

Preferably, the bottom of the filter cartridge and the atomizer body are jointly pressed against the large-sized O-ring to deform and form a seal.

Preferably, the atomizing body assembly further contains a shim for the T-shaped steel member. The shim is mounted between the large metal cap and the T-shaped steel member.

In some embodiments, the material of the chip-mounting part may be selected from elastically deformable materials, such as silicone, rubber, TPE, TPU, and any other similar materials. Preferably, the silicone material is selected in this application.

A soft mist device is installed with an atomizing body assembly. The atomizing body assembly is capable of improving sealing performance.

Certain advantages of the present disclosure include:

By setting a small conical surface and a large conical surface on the top of the chip-mounting part, the degree of deformation is enhanced, thereby improving the clamping performance on the chip and further improving the sealing performance.

The bottom of the chip-mounting part is provided with an annular structure protruding radially outward, so that the end surface of the T-shaped steel member may be pressed against the step surface of the chip-mounting part and cause deformation of the medium-sized O-ring. The deformed medium-sized O-ring may form a seal between the bottom surface of the chip-mounting part and the filter cartridge including the top annular surface.

### BRIEF DESCRIPTION OF THE DRAWINGS

FIG. 1 shows a diagram of a soft mist device.

FIG. 2 shows a diagram of an atomizing body assembly.

FIG. 3 shows an exploded diagram of the sealing structure in the atomizing body assembly.

FIG. 4 shows an axonometric view of a T-shaped steel member in one direction.

FIG. 5 shows an axonometric view of the T-shaped steel member in another direction.

FIG. 6 shows an axial cross-sectional view of the T-shaped steel member in one direction.

FIG. 7 shows an axial cross-sectional view of the T-shaped steel member in another direction.

FIG. 8 shows an axonometric view of a shim for the T-shaped steel member.

FIG. 9 illustrates an axial cross-sectional view of a chip-mounting part in one direction.

FIG. 10 illustrates an axial cross-sectional view of the chip-mounting part in another direction.

FIG. 11 shows an axonometric view of the chip-mounting part in yet another direction.

FIG. 12 illustrates an axonometric view of the chip-mounting part in yet another direction.

FIG. 13 illustrates an axonometric view of a chip in one direction.

FIG. 14 illustrates an axonometric view of a chip in another direction.

FIG. 15 illustrates an axial cross-sectional view of a chip in one direction.

FIG. 16 illustrates an axial cross-sectional view of a chip in another direction.

FIG. 17 illustrates an axial cross-sectional view of a chip mounted in the chip-mounting part in one direction.

FIG. 18 illustrates an axial cross-sectional view of a chip mounted in a chip-mounting part in another direction.

FIG. 19 shows an axial cross-sectional view of a chip mounted in the chip-mounting part, which is mounted in a T-shaped steel member in one direction.

FIG. 20 shows an axonometric view of the filter cartridge.

FIG. 21 shows an axial cross-sectional view along an axis of the filter cartridge.

FIG. 22 shows an axial cross-sectional view of the medium-sized O-ring and the filter element which are mounted on the filter cartridge in one direction.

FIG. 23 shows a view before preliminary loading.

FIG. 24 shows a view of medicinal liquid being sprayed out as aerosols after preliminary loading.

### DETAILED DESCRIPTION OF THE INVENTION

In order to describe the application, references to embodiments and/or methods of the application will be listed in detail. One or more examples, together with the drawings, are used to illustrate the application. Each example is provided in such a way as to explain the application and is not intended to define the application. Indeed, for persons skilled in the art, other embodiments may be conceived by various modifications and changes within the scope and spirit of the application. For example, features or steps shown or described as part of one embodiment may be used with another embodiment or step to produce further embodiments or methods. The scope of protection of the present application is therefore intended to cover such modifications and changes, which are within the scope of the claims and their equivalents.

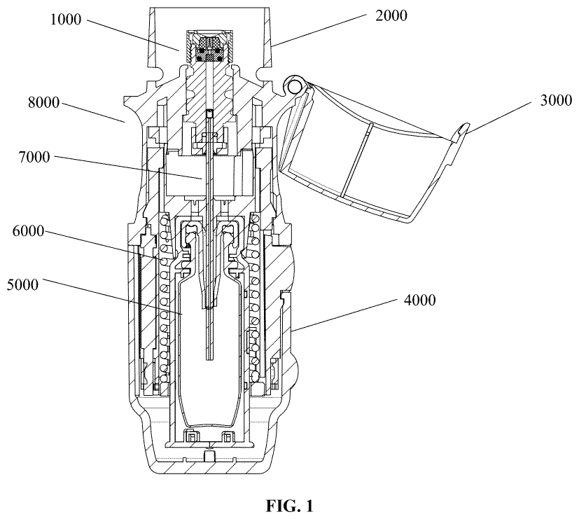

In order to facilitate understanding, orientation words such as “upper” and “lower”, “left” and “right”, “front” and “back” might be obtained intuitively from the drawings of the specification. When there is a conflict between the orientation words of two attached drawings, the orientation presented in the assembly diagram of FIG. 1 shall prevail.

FIG. 1 shows a soft mist device comprising an atomizing body assembly 1000, a mouthpiece 2000, an upper cover 3000, a transparent cover 4000, a bottle 5000 for holding medicinal liquid, a spring 6000, a capillary assembly 7000, and an atomizer housing 8000.

The atomizer housing 8000 is the main housing component of the soft mist device, providing protection and support to the entire soft mist device. The atomizer housing 8000 is divided into an upper part and a lower part, which are capable of rotating relative to each other.

A mouthpiece 2000 is provided at the top of the upper part of the atomizer housing 8000. When the patients cover the mouthpiece 2000 with their mouths, the medicinal liquid can be sprayed as aerosols, which may completely enter the patient's mouth.

The atomizing body assembly 1000 is provided within the upper end of the upper part of the atomizer housing 8000. Because of the structure of atomizing body assembly 1000, the medicinal liquid is discharged as aerosols from the upper end of the atomizing body assembly 1000. The lower end of the atomizing body assembly 1000 is provided with a capillary assembly 7000.

The capillary assembly 7000 is disposed inside the atomizer housing 8000. The upper end of the capillary assembly 7000 is disposed at the lower end of the atomizing body assembly 1000. The lower end of the capillary assembly 7000 is inserted into the bottle 5000 for holding medicinal liquid. The capillary assembly 7000 sucks the medicinal liquid from the bottle 5000 and then delivers into the atomizing body assembly 1000, providing a continuous supply of the medicinal liquid to be atomized as long as a sufficient amount of the medicinal fluid is left in the bottle 5000.

The bottle 5000 is disposed inside the atomizer housing 8000. The upper end of the bottle 5000 is fixed on the sliding structure of the atomizer housing 8000, and the bottle 5000 is used to store the medicinal liquid to be nebulized. In order to save materials, a soft mist device allowing a patient to replace the bottle 5000 has been developed.

The spring 6000 is disposed inside the atomizer housing 8000. The upper end of the spring 6000 is fixed to the sliding structure of the upper part of atomizer housing 8000, and the lower end of the spring 6000 is fixed to the lower part of the atomizer housing 8000. The sliding structure of the upper part of the atomizer housing 8000 can be axially moved when the upper part and the lower part may rotate relative to each other, thereby driving the spring 6000 to elongation or compression. This elongation or compression provides power for the medicinal liquid to become an aerosol.

An upper cover 3000 is provided on one side edge of the upper end of the upper part of the atomizer housing 8000. One side of upper cover 3000 is rotatably fixed. When the patients do not use the soft mist device, the upper cover 3000 covers the mouthpiece 2000 so that the mouthpiece 2000 does not come into contact with external objects when the soft mist device is carried by the patient, thereby preventing the mouthpiece 2000 from being contaminated. At this time, the other side of the upper cover 3000 is fastened on the other side edge of the upper end of the upper part of the atomizing body assembly 1000 in a snap-fit manner. When the patients need to use the soft mist device, thanks to the snap-fit fastening, the patients can easily release the snapped state and rotate the upper cover 3000 to expose the mouthpiece 2000.

A transparent cover 4000 is provided at the outside of the lower end of the lower part of the atomizer housing 8000. In one aspect, the transparent cover 4000 prevents external objects from entering the lower end of the atomizer housing 8000, and in another aspect, it is convenient for the patient to clearly see how many times the soft mist device has been used or how much liquid remains in the soft mist device. The transparent cover 4000 is snap-fitted to the outside of the lower end of the atomizer housing 8000.

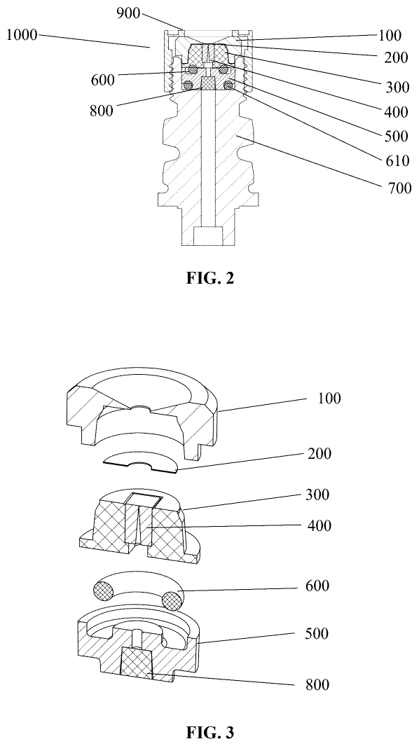

FIGS. 2 and 3 illustrate a schematic structural diagram of an atomizing body assembly 1000. The atomizing body assembly 1000 includes a T-shaped steel member 100, a shim 200 for the T-shaped steel member, a chip-mounting part 300, a chip 400, a filter cartridge 500, a medium-sized O-ring 600, a large-sized O-ring 610, an atomizer body 700, a filter element 800, and a large metal cap 900. These components cooperate with each other to form a sealed atomizing system.

Internal threads are provided on the inner wall of the lower end of the large metal cap 900. External threads are provided on the outer wall of the upper end of the atomizer body 700. The large metal cap 900 is threadedly connected to the atomizer body 700 by means of internal threads of the large metal cap 900 and external threads of the atomizer body 700. The large metal cap 900 is threadedly fixed to the upper end of the atomizer body 700. The nebulizer body 700 and the large metal cap 900 are assembled together to form a receiving cavity. The receiving cavity is used to install the T-shaped steel member 100, the shim 200, the chip-mounting part 300, the chip 400, the filter cartridge 500, the medium-sized O-ring 600, the large-sized O-ring 610, and the filter element 800.

A recess is provided on the upper end of the atomizer body 700. The recess is used to accommodate filter cartridge 500, medium-sized O-ring 600, large-sized O-ring 610, and filter element 800. The recess is also used to position the chip 400, the T-shaped steel member 100, the shim 200, and the chip-mounting part 300. The upper annular surface at the bottom of the recess contacts the bottom annular surface of the filter cartridge 500. The inner cylindrical surface of the recess contacts the outer cylindrical surface of the filter cartridge 500. The center position of the bottom annular surface of the filter cartridge 500 is provided with a filter receiving chamber for mounting the filter element 800. The top annular surface of the filter cartridge 500 contacts the lower annular surface at the bottom of the chip-mounting part 300. The outer cylindrical surface at the bottom edge of the chip-mounting part 300 contacts the inner cylindrical surface of the recess. The top of the chip-mounting part 300 is provided with a groove for positioning the chip 400. The top of the chip-mounting part 300 is accommodated in a receiving cavity of the T-shaped iron piece 100. The outer cylindrical surface of the lower end of the receiving cavity of the T-shaped steel member 100 contacts the inner cylindrical surface of the recess of the atomizer body 700. The annular surface on the top of the T-shaped steel member 100 contacts the large metal cap 900.

The top of the filter cartridge 500 is provided with an annular recess for positioning the medium-sized O-ring 600. The bottom of the filter cartridge 500 is provided with another annular recess for positioning the large-sized O-ring 610. In order to further adjust the sealing effect, the shim 200 is provided at the contact position between the end of the top of the chip-mounting part 300 and the inside of the receiving cavity of the T-shaped steel member 100. The shim 200 includes an annular structure.

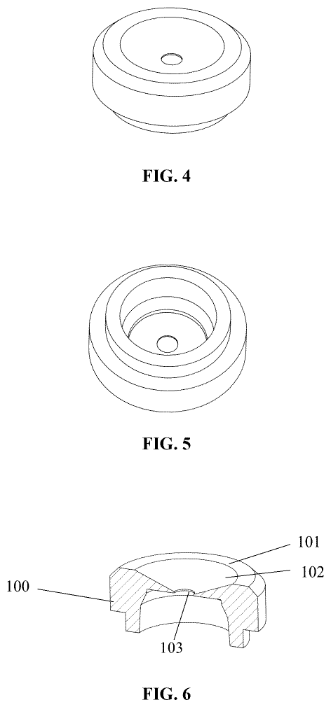

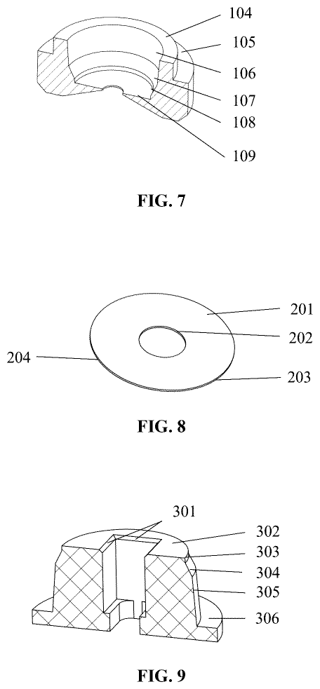

FIG. 4, FIG. 5, FIG. 6, and FIG. 7 show axonometric and cross-sectional views of a T-shaped steel member 100 including a top surface 101, a conical surface 102, a hole 103, an end surface 104, a step surface 105, a large conical surface 106, a small conical surface 107, a cylindrical surface 108, and a top surface 109. The T-shaped steel member 100 is pressed against the chip-mounting part 300 to form a seal on the top of the chip.

The T-shaped steel member 100 has a “T” shape and a cylindrical housing structure. The top surface 101 is provided at the top of the T-shaped steel member 100. The top surface 101 has an annular top surface. The middle of the top surface 101 is provided with a conical surface 102. The center of the conical surface 102 is provided with a hole 103, and the hole 103 includes a form of through-hole. The aerosols are sprayed from the hole 103 and then released from the soft mist device. The edge of the top end of the T-shaped steel member 100 includes a cylindrical surface. The upper end of the cylindrical surface of the top of the T-shaped steel member 100 is connected to the top surface 101 through a chamfering transition. The lower end of the cylindrical surface of the top of the T-shaped steel member 100 is provided with a step surface 105. The bottom of the step surface 105 is connected to the bottom of the T-shaped steel member 100. The bottom of the T-shaped steel member 100 has a cylindrical structure. The bottom of the T-shaped steel member 100 is provided with an end surface 104. The outer diameter of the bottom of the T-shaped steel member 100 is smaller than the outer diameter of the top of the T-shaped steel member 100. The end surface 104 is used to contact the step surface 306 of the chip-mounting part 300.

The T-shaped steel member 100 has a “T” shape and a cylindrical housing structure. Inside the T-shaped steel member 100, a large conical surface 106 is sequentially connected with a small conical surface 107, a cylindrical surface 108, and a top surface 109. The large conical surface 106, the small conical surface 107, the cylindrical surface 108, and the top surface 109 form a receiving cavity for accommodating the top of the chip-mounting part 300.

FIG. 8 shows a diagram of shim 200 for the T-shaped steel member. The shim 200 has an annular structure. Here, it should be noted that the shim 200 is optionally included. The shim 200 allows the sealing space formed by the atomizing body assembly 1000 to be adjustable. In order to show the specific structure of the shim 200, the present disclosure will be described in embodiments where the shim 200 is used. The shim 200 includes a top annular surface 201, a cylindrical hole surface 202, an outer cylindrical surface 203, and a bottom annular surface 204. The top annular surface 201 fits with the top surface 109 of the T-shaped steel member 100. The outer cylindrical surface 203 is clearance-fitted with the cylindrical surface 108 of the T-shaped steel member 100. The bottom annular surface 204 is pressed against the top surface 302 of the chip-mounting part.

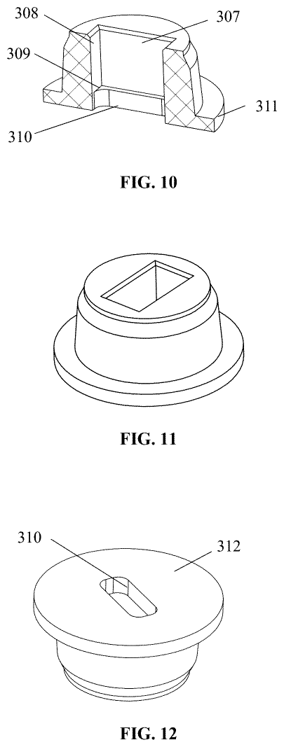

FIG. 9, FIG. 10, FIG. 11, and FIG. 12 show cross-sectional and axonometric views of a chip-mounting part 300. The chip-mounting part 300 includes an elastically deformable body that can be deformed by pressure. The chip-mounting part 300 can be made of elastically deformable material, such as silicone, rubber, TPE, TPU, and any other similar materials. In some embodiments, the material selected by the present disclosure includes silicone.

The chip-mounting part 300 includes an inclined surface 301, a top surface 302, a cylindrical surface 303, a small conical surface 304, a large conical surface 305, a step surface 306, a surface 307, a surface 308, a surface 309, a hole 310, a side 311, and a bottom surface 312. The chip-mounting part 300 includes a cylindrical structure with an annular structure protruding radially outward at the bottom. The top of chip-mounting part 300 is provided with top surface 302. Starting from the top surface 302, the cylindrical surface 303, the small conical surface 304, the large conical surface 305, and the step surface 306 are connected sequentially. The step surface 306 corresponds to an upper annular surface of the annular structure protruding radially outward at the bottom of the chip-mounting part 300. The lower annular surface of the annular structure protruding radially outward at the bottom of the chip-mounting part 300 and a bottom end surface of the chip-mounting part 300 are disposed on the same plane and form a bottom surface 312.

The chip-mounting part 300 also includes a cylindrical housing structure. The chip-mounting part 300 has a through-hole penetrating therethrough along the axis of chip-mounting part 300. The through-hole includes a groove for accommodating the chip 400 and a hole 310. The groove is connected with the hole 310. The cross-section of the hole 310 is smaller than the cross-section of the groove. The longest dimension in each direction of the cross section of the hole 310 is less than or equal to the longest dimension in each corresponding direction of the cross section of the groove. For the convenience of understanding, the hole 310 shown in the drawings of the present disclosure includes a slot-shaped hole. In the slot-shaped hole, the longitudinal length of the slot is nearly the same length as the surface 307. The groove for accommodating the chip 400 includes four inclined surfaces 301, two surfaces 307, two surfaces 308 and a surface 309. The groove containing the chip 400 includes a groove with chamfered cuboid space. The inclined surface 301 facilitates a smooth insertion of the chip 400 into the groove.

The cylindrical surface 303, the small conical surface 304 and the large conical surface 305 of the chip-mounting part 300 respectively have an interference-fit with the cylindrical surface 108, the small conical surface 107 and the large conical surface 106 of the T-shaped steel member 100. Under the interference-fit, the chip-mounting part 300 and the chip 400 are pressed against each other to achieve the purpose of sealing the chip. Upon the installation of the T-shaped steel member 100 with the chip-mounting part 300, the step surface 306 and the end surface 104 are pressed against each other, and then the medium-sized O-ring 600 is pressed.

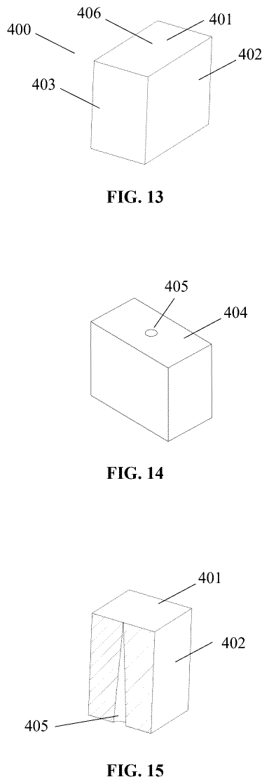

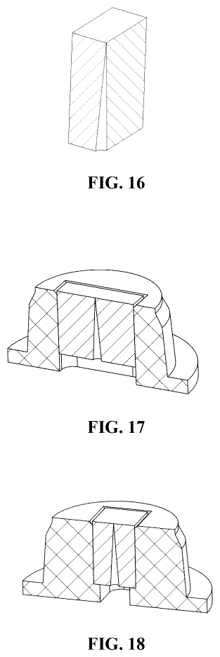

FIG. 13, FIG. 14, FIG. 15, and FIG. 16 illustrate axonometric and cross-sectional views of a chip 400. The chip 400 is formed by attaching two plates together. Between the two contact surfaces of the plates that are stacked together in a face-to-face manner, one contact surface is provided with a micro-fluidic channel and the other contact surface is smooth and flat. When the two plates are attached together, the medicinal liquid can only pass through the micro-fluidic channel and then get sprayed as an aerosol with a size of about 1-5 microns.

The chip 400 includes a cuboid structure including a top surface 401, a side 402, a side 403, a bottom surface 404, a liquid inlet 405, and a liquid outlet 406. The end surfaces of the top surface 401 and the bottom surface 404 are both rectangular. The two sides 402 and the two sides 403 are arranged around the top surface 401 and the bottom surface 404. The liquid inlet 405 is disposed on the bottom surface 404, and the liquid outlet 406 is disposed on the top surface 401.

The top surface 401 of the chip 400 is in contact with the top surface 109 of the T-shaped steel member 100. In other embodiments, the top surface 401 of the chip 400 contacts the bottom annular surface 204 of the shim 200, and the top annular surface 201 of the shim 200 contacts the top surface 109 of the T-shaped steel member 100. The side 402 and side 403 of the chip 400 have an interference fit with the surfaces 307 and surface 308 of the chip-mounting part 300, respectively, to seal the chip. The bottom surface 404 of the chip 400 cooperates with the surface 309 at the bottom of the groove for accommodating and supporting the chip 400.

FIGS. 17 and 18 show cross-sectional views of the assembly of chip 400 and the chip-mounting part 300 in two different directions. The installation process of the chip 400 has directional requirements. The liquid inlet 405 is set on the bottom surface 404 of the chip 400 and should be installed at the bottom. The liquid outlet 406 is set on the top surface 401 of the chip 400 and should be installed at the top. When the medicinal liquid passes through the hole 310 of chip-mounting part 300, the medicinal liquid passes through the inlet 405, goes through the micro-fluidic channel, becomes aerosols of about 1-5 microns, and then is sprayed from the liquid outlet 406.

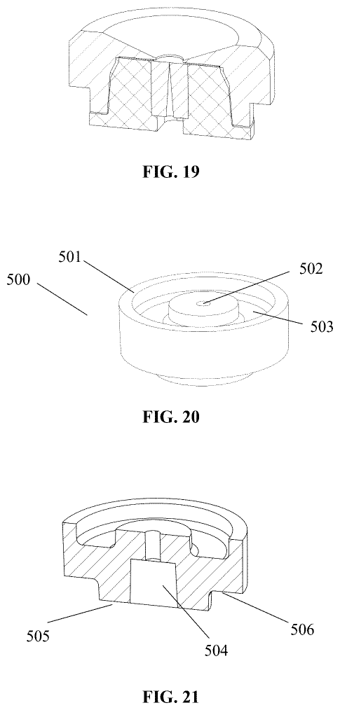

FIG. 19 shows a cross-sectional view of an assembly including the T-shaped steel member 100, the chip-mounting part 300, and the chip 400. When the chip 400 is installed in the chip-mounting part 300 in accordance with the directional requirements, then chip-mounting part 300 is installed in the receiving cavity of the T-shaped steel member 100. The chip-mounting part 300 is partially housed in the receiving cavity of the T-shaped steel member 100. The top of the chip-mounting part 300 is housed in the receiving cavity of the T-shaped steel member 100. The bottom of the chip-mounting part 300 is not housed in the receiving cavity of the T-shaped steel member 100. The step surface 306 of the chip-mounting part 300 is in contact with the end surface 104 of the T-shaped steel member100. After the soft mist device is assembled and completed, the step surface 306 of the chip-mounting part 300 is pressed against the end face 104 of the T-shaped steel member 100, thereby forming a seal.

Regarding the diameter size of the cross-section of the bottom portion of the chip-mounting part 300, it may be the same as that of the end face 104 of the T-shaped steel member 100 or it may be different from the cross-section of the end face 104 of the T-shaped steel member 100. However, no matter what diameter is determined, it must be ensured that the bottom of the chip-mounting part 300 is provided with the step surface 306, and the step surface 306 is in contact with the end face 104 of the T-shaped steel member 100. For the sake of an aesthetic and simplified design, it is shown in the drawings of the present disclosure that the diameter size of the cross section of the bottom portion of the chip-mounting part 300 is the same as the diameter size of the cross section of the end face 104 of the T-shaped steel member 100.

Meanwhile, for other possible technical options, the shim 200 can be mounted within the receiving cavity of the T-shaped steel member 100, thereby allowing an adjustable sealing performance.

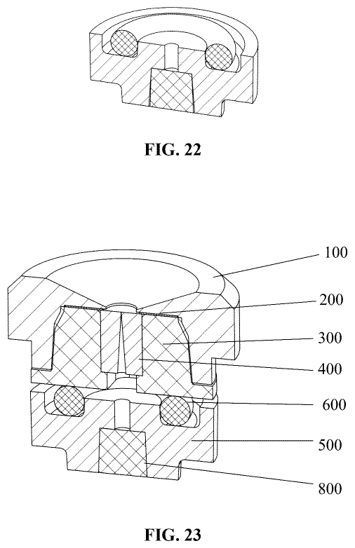

FIG. 20, FIG. 21, and FIG. 22 illustrate axonometric and axial cross-sectional views of a filter cartridge 500. The filter cartridge 500 includes a cylindrical structure with an annular groove and a through-hole along the axis. The filter cartridge 500 includes a top annular surface 501, a bottom annular surface 505, a top annular groove 503, a bottom annular groove 506, and a through-hole. The top annular groove 503 is disposed on the top annular surface 501, both of which are concentric to each other. The bottom annular groove 506 is disposed on the bottom annular surface 505, both of which are concentric to each other. The through-hole of the filter cartridge 500 includes a cylindrical hole 502 and a conical hole 504. The diameter of the cylindrical hole 502 is smaller than that of the conical hole 504. The cylindrical hole 502 extends downward from the top annular surface 501. The conical hole 504 extends upward from the bottom annular surface 505. The cylindrical hole 502 and the conical hole 504 is in a fluid communication with each other. A medium-sized O-ring 600 is disposed in the top annular groove 503. A large-sized O-ring 610 is accommodated in the bottom annular groove 506. The conical hole 504 is used to accommodate the filter element 800.

FIG. 23 shows a view before a preliminary loading. The filter cartridge 800 is assembled into the conical hole 504. The medium-sized O-ring 600 is disposed in the top annular groove 503. The chip-mounting part 300 is mounted with the chip 400, and after being assembled, both of them are accommodated in the T-shaped steel member 100. The filter cartridge 500 and the T-shaped steel member 100 are assembled together, and after assembly, both of them are disposed in the recess at the upper end of the atomizer body 700.

Diameters are the same regarding the diameter size of the largest cross section of the cylindrical structure for the filter cartridge 500, the diameter size of the cross section of the bottom portion of the chip-mounting part 300, and the diameter size of the cross section of the end face 104 of the T-shaped steel member 100.

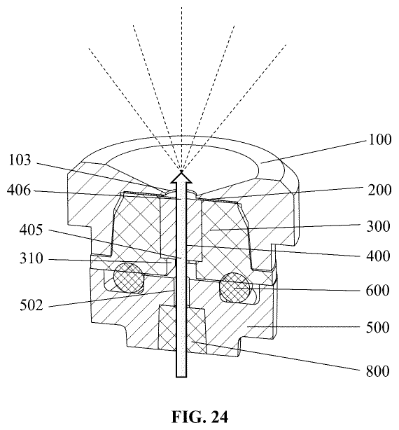

FIG. 24 illustrates an assembly diagram of the large metal cap 900 and the atomizer body 700 after tightening. The filter cartridge 500 and the T-shaped steel member 100 are assembled and together are installed in a recess at the upper end of the nebulizer body 700. Then the large metal cap 900 is installed and the cavity of large metal cap 900 covers T-shaped steel member 100. The large metal cap 900 is tightened to the upper portion of the atomizer body 700, where it has been described previously that the large metal cap 900 and the atomizer body 700 are threadedly connected. During the tightening process, the large metal cap 900 is pressed against the T-shaped steel member 100, and the receiving cavity of the T-shaped steel member 100 acts on the cylindrical surface 303, the small conical surface 304 and the large conical surface 305 of the chip-mounting part 300 through the cylindrical surface 108, the small conical surface 107 and the large conical surface 106, respectively, so that the chip-mounting part 300 is deformed to clamp the chip 400. At the same time, the end surface 104 of the T-shaped steel member 100 is pressed against the step surface 306 of the chip-mounting part 300 and accordingly the bottom surface 312 of the chip-mounting part 300 contacts the top annular surface 501 of the filter cartridge 500. The contact between the bottom surface 312 and the top annular surface 501 causes the deformation of medium-sized O-ring 600, so that a seal is formed between the bottom surface 312 of the chip-mounting part 300 and the top annular surface 501 of the filter cartridge 500. At the same time, the filter cartridge 500 is pressed against the large-sized O-ring 610, and accordingly the large-sized O-ring 610 is deformed to form a seal between the bottom annular surface 505 of the filter cartridge 500 and the bottom of the recess at the upper end of the atomizer body 700.

When the large metal cap 900 is tightened to a predetermined position, the chip 400 is firmly fixed within the chip-mounting part 300. A seal is formed between the bottom surface 312 of the chip-mounting part 300 and the top annular surface 501 of the filter cartridge 500. Another seal is also formed between the bottom annular surface 505 of the filter cartridge 500 and the bottom portion of the recess at the upper end of the atomizer body 700. The medicinal liquid passes through the filter element 800 and then sequentially flows through the cylindrical hole 502 of the filter cartridge 500, the slot-shaped hole 310 at the bottom of the chip-mounting part 300, the liquid inlet 405 of the chip 400, and then flows through the micro-fluidic channel of the chip 400 to become aerosols with a size of about 5 microns. It is also meant that the medicinal liquid is sprayed from the liquid outlet 406.

The present application enhances the clamping effect on the chip 400 by providing a small conical surface 304 and a large conical surface 305 on the top of the chip-mounting part 300, thereby enhancing the deformation degree of chip-mounting part 300 and further improving the sealing performance to clamp the chip 400. At the same time, the chip-mounting part 300 is provided with an annular structure protruding radially outward at the bottom, so that the end surface 104 of the T-shaped steel member 100 may be pressed against the step surface 306 of the chip-mounting part 300, thus causing the medium-sized O-ring 600 to deform. Another seal is also formed between the bottom surface 312 of the chip-mounting part 300 and the top annular surface 501 of the filter cartridge 500.

Although various embodiments of the disclosure have been described above, it should be appreciated that they are presented by example only and the scope of the disclosure is not limited to these embodiments. For example, the disclosure is not limited to the physical layout or size which is described or illustrated. The application is also not limited to any particular design or material. Thus, the breadth and scope of the present disclosure should not be limited to any of the above exemplary embodiments, but should be defined only in accordance with the claims and their equivalents.

What is claimed is:

1-17. (canceled)

18. An atomizing body assembly (1000), comprising:
a T-shaped steel member (100),
a chip-mounting part (300),
a filter cartridge (500),
a medium-sized O-ring (600);
wherein:
a chip (400) is disposed within the chip-mounting part (300), the chip-mounting part (300) has a top portion mounted within a receiving cavity of the T-shaped steel member (100) to fixedly secure and seal the chip (400), a bottom of the chip-mounting part (300) contacts a top portion of the filter cartridge (500);
the medium-sized O-ring (600) is interposed between the chip-mounting part (300) and the filter cartridge (500), and
the chip-mounting part (300), the medium-sized O-ring (600) and the filter cartridge (500) collectively form a sealed structure.

19. The atomizing body assembly according to claim 18, wherein the T-shaped steel member (100) comprises a cylindrical housing structure having sequentially connected internal surfaces defining said receiving cavity,
the internal surfaces comprising:
a large conical surface (106),
a small conical surface (107),
a cylindrical surface (108), and
a top surface (109).

20. The atomizing body assembly according to claim 18, wherein the chip-mounting part (300) includes a cylindrical structure with an annular structure protruding radially outward at its bottom portion.

21. The atomizing body assembly according to claim 18, wherein the T-shaped steel member (100) comprises an end face (104), and the chip-mounting part (300) comprises a step surface (306), and wherein the end face (104) of the T-shaped steel member (100) is pressed against the step surface (306) of the chip-mounting part (300).

22. The atomizing body assembly according to claim 18, wherein the filter cartridge (500) comprises:
a cylindrical body with two annular grooves and an axial through-hole.

23. The atomizing body assembly according to claim 18, wherein the filter cartridge (500) comprises:
a top annular surface (501),
a bottom annular surface (505),
a top annular groove (503),
an axial through-hole, and
an annular groove (506);
wherein the top annular groove (503) is disposed on the top annular surface (501) and
the annular groove (506) is disposed on the bottom annular surface (505).

24. The atomizing body assembly according to claim 18, further comprising:
a large-sized O-ring (610), and
an atomizer body (700),
wherein a bottom portion of the filter cartridge (500) and the atomizer body (700) presses the large O-ring (610) to cause deformation and form a seal.

25. The atomizing body assembly according to claim 18, further comprising a shim (200) and a large metal cap (900); and
wherein the shim (200) is disposed between the large metal cap (900) and the T-shaped steel member (100).

26. The atomizing body assembly according to claim 18, wherein the chip-mounting part (300) comprises an elastically deformable material, and
wherein the elastically deformable material is silicone, rubber, TPE or TPU.

27. The atomizing body assembly according to claim 20, wherein the chip-mounting part (300) comprises the following surfaces connected sequentially from top to bottom:
a top surface (302),
a cylindrical surface (303),
a small conical surface (304),
a large conical surface (305), and
a step surface (306) forming an upper annular surface of the annular structure protruding radially outward.

28. The atomizing body assembly according to claim 20, wherein a lower upper annular surface of the annular structure protruding radially outward and a bottom end surface of the chip-mounting part (300) collectively define a bottom surface (312); and
the bottom surface (312) is pressed against a top portion of the filter cartridge (500).

29. The atomizing body assembly according to claim 20, wherein the chip-mounting part (300) comprises a through-hole disposed along an axis of the chip-mounting part (300), the through-hole comprising:
a chip-receiving groove for accommodating the chip (400), and a hole (310), the chip-receiving groove being in a fluid communication with the hole (310).

30. The atomizing body assembly according to claim 22, wherein:
the chip-mounting part (300) comprises a bottom surface (312),
the filter cartridge (500) comprises a top annular surface (501) and a top annular groove (503),
the medium-sized O-ring (600) is disposed in the top annular groove (503),
the bottom surface (312) is pressed against on both the medium-sized O-ring (600) and the top annular surface (501) of the filter cartridge (500), and
deformation of the chip-mounting part (300) and the medium-sized O-ring (600) enhances sealing performance.

31. The atomizing body assembly according to claim 25, further comprising:
a large-sized O-ring (610),
an atomizer body (700), and
a large metal cap (900) threadedly engaged with the atomizer body (700) to define an accommodating cavity that houses:
the T-shaped steel member (100),
the chip-mounting part (300),
the chip (400),
the filter cartridge (500),
the medium-sized O-ring (600), and
the large-sized O-ring (610);
wherein:
the T-shaped steel member (100) comprises a top portion,
the filter cartridge (500) comprises a bottom annular surface (505) and a bottom annular groove (506);
an inner surface of the large metal cap (900) is pressed against the top portion of the T-shaped steel member (100),
the atomizer body (700) is pressed against the bottom annular surface (505) of the filter cartridge (500), and
the large-sized O-ring (610) is disposed in the bottom annular groove (506) of the filter cartridge (500).

32. The atomizing body assembly according to claim 29, wherein the hole (310) comprises a slot-shaped hole.

33. The atomizing body assembly according to claim 29, wherein the chip-receiving groove comprises an inclined surface (301) at its opening to facilitate a smooth insertion of the chip (400).

34. A soft mist device comprising: an atomizing body assembly (1000) as claimed in claim 18.
